(19) United States
(12) Patent Application Publication
(10) Pub. No.: US 2018/0311375 A1
(43) Pub. Date: Nov. 1, 2018

(54) PROCESS OF PREPARING ANTIBODY-DRUG CONJUGATE

(71) Applicant: CADILA HEALTHCARE LIMITED, Ahmedabad (IN)

(72) Inventors: Sanjeev Kumar MENDIRATTA, Ahmedabad (IN); Sanjay BANDYOPADHYAY, Ahmedabad (IN); Avanish Kumar SINGH, Ahmedabad (IN); Umang TRIVEDI, Ahmedabad (IN); Ajinath TAMBE, Ahmedabad (IN)

(21) Appl. No.: 15/963,733

(22) Filed: Apr. 26, 2018

(30) Foreign Application Priority Data
Apr. 27, 2017 (IN) 201721014917

(51) Int. Cl. A61K 47/68; C07K 1/18; C07K 1/34; C07K 16/32; A61K 31/537

(52) U.S. Cl. CPC A61K 47/6851; C07K 1/18; C07K 1/34; C07K 2317/24; A61K 31/537; A61K 47/6803; A61K 47/6889; C07K 16/32

(57) ABSTRACT

The invention provides process for preparing antibody-drug conjugates, preferably trastuzumab maytansinoid conjugate linked via non-cleavable linker comprising performing the conjugation reaction at lower temperature than ambient and/or at lower pH condition than neutrality. The process according to the present invention comprises conjugating the linker modified antibody with cytotoxic drug, preferably the SMCC-modified trastuzumab with a cytotoxic drug, preferably maytansinoid at a temperature lower than 20° C. and at a pH lower than 6.0, and thereby preparing an antibody-drug conjugate, preferably trastuzumab maytansinoid conjugate, T-DM1.

### FIELD OF THE INVENTION

The invention provides process for preparing antibody-drug conjugates, preferably trastuzumab maytansinoid conjugate linked via non-cleavable linker comprising performing the conjugation reaction at lower temperature than ambient and/or at lower pH condition than neutrality. The process according to the present invention comprises conjugating the linker modified antibody with cytotoxic drug, preferably the SMCC-modified trastuzumab with a cytotoxic drug, preferably a maytansinoid at a temperature lower than 20° C. and at a pH lower than 6.0, and thereby preparing an antibody-drug conjugate, preferably trastuzumab maytansinoid conjugate, T-DM1.

### BACKGROUND OF THE INVENTION

The treatment of cancer has progressed significantly with the development of pharmaceuticals that more efficiently target and kill the cancer cells. To this end, researchers have been able to develop therapeutic monoclonal antibodies that bind to the tumor-specific or tumor-associated antigens on cancer cells. Further, cytotoxic molecules such as bacteria and plant toxins, radionuclides, and certain chemotherapeutic drugs have been employed to form monoclonal antibody-drug conjugate (chemically linked) which binds to the tumor-specific or tumor-associated cell surface antigens (see, e.g., International Patent Applications WO 00/02587, WO 02/060955, and WO 02/092127, U.S. Pat. Nos. 5,475,092, 6,340,701, and 6,171,586) and causes cancer cell death. Such cytotoxic drug conjugated form of antibody molecules are typically referred to as immunoconjugates or radioimmunoconjugates or immunotoxins. Cell killing occurs through binding of the antibody-drug conjugate to a tumor cell followed by its internalization and release and/or activation of the cytotoxic activity of the drug. The selective delivery of the toxin drug through antibody-drug conjugated complex minimizes exposure of the normal cells to the toxin drugs leading to enhanced tolerability of the drug in patients.

Processes for conjugating antibodies to sulfhydryl-containing cytotoxic drugs such as maytansinoids have been described previously (see, e.g., U.S. Pat. Nos. 5,208,020, 5,416,064, and 6,441,163). For example, U.S. Pat. Nos. 5,208,020 and 5,416,064 disclose a process for manufacturing antibody-maytansinoid conjugates, wherein the antibody is first modified with a heterobifunctional reagent as described in U.S. Pat. Nos. 4,149,003, 4,563,304 and U.S. Patent Application Publication No. 2004/0241174 A1. Further, conjugation of a modified antibody with sulfhydryl-containing cytotoxic drug at pH 7 and purification of the drug conjugated antibody by gel filtration chromatography has been described in U.S. Pat. Nos. 5,208,020 and 5,416,064. Purification of antibody-drug conjugates by size exclusion chromatography (SEC) has been described [see, e.g., Liu et al., Proc. Natl. Acad. Set (USA), 93: 8618-8623 (1996), and Chari et al., Cancer Research, 52: 127-131 (1992)].

Commonly used manufacturing processes comprise a modification step, in which a bifunctional linker is covalently attached to antibody at room temperature (about 20° C.) or above leaving the other reactive group of the linker molecule intact. Following modification, the antibody undergoes a conjugation reaction with a cytotoxic drug at room temperature (about 20° C.) or above to form the drug conjugated antibody with the formation of a covalent chemical linkage between the linker (using the reactive group) and the cytotoxic drug.

It would be desirable to modify one or more process steps without any significant change in purity or functional properties or stability of the desired product. It would be further desirable to obtain higher level of purity through additional purification step(s) than those that have been so far described in literature. In that case, some of the purification procedure(s) might be more efficacious to isolate and purify the desired molecule having certain combinations of cell binding agents, linkers, and drugs than the others.

In view of the foregoing, there is a need in the art to develop improved methods of preparing drug conjugated antibody compositions with substantially high level of purity and adequate stability. The present invention provides such a method. These and other advantages of the invention as well as additional inventive features are described herein this application. WO 2007024536 provides a process for preparing a cell-binding agent-drug conjugate comprising the steps of: (a) contacting a cell-binding agent with a bifunctional crosslinking reagent to covalently attach a linker to the cell-binding agent and thereby prepare a first mixture comprising cell-binding agents having linkers bound thereto, (b) subjecting the first mixture to tangential flow filtration, selective precipitation, adsorptive filtration, or an adsorptive chromatography resin and thereby prepare a purified first mixture of cell-binding agents having linkers bound thereto, (c) conjugating a drug to the cell-binding agents having linkers bound thereto in the purified first mixture by reacting the cell-binding agents having linkers bound thereto with a drug in a solution having a pH of about 4 to about 9 to prepare a second mixture comprising (i) cell-binding agent chemically coupled through the linker to the drug, (ii) free drug, and (iii) reaction by-products, and (d) subjecting the second mixture to a tangential flow filtration, selective precipitation, adsorptive filtration, or an adsorptive chromatography resin to purify the cell-binding agents chemically coupled through the linkers to the drug from the other components of the second mixture and thereby prepare a purified second mixture.

WO 2012135522 discloses a process for preparing a cell-binding agent having a linker bound thereto, which process comprises contacting a cell-binding agent with a bifunctional crosslinking reagent at a temperature of about 15° C. or less to covalently attach a linker to the cell-binding agent and thereby prepare a mixture comprising cell-binding agents having linkers bound thereto.

WO 2014055842 provides processes for manufacturing cell-binding agent-cytotoxic agent conjugates of improved stability comprising performing the modification reaction at a high pH.

None of these published literature discloses a process of making cytotoxic drug conjugating to the linker modified antibody where conjugation of the drug to the modified antibody is performed at a temperature lower than 20° C. and at a pH lower than 6.0, as disclosed, herein. Further, in the manufacture of therapeutic proteins, aggregation is a common problem. Protein aggregates such as high molecular weight species (HMW species), Low molecular weight species (LMW species) etc. have the potential to affect the safety and efficacy of biologic drug substance. HMW species are protein aggregates includes dimers, trimers, tetramers, etc. formed of monomers that can be either covalently or non-covalently linked. Protein aggregates can be categorized in several ways, including soluble/insoluble, covalent/non-covalent, reversible/non-reversible, and native/denatured. These structural changes are significant because they can cause a loss of activity of the intact proteins. Furthermore, aggregation and misfolding can induce a new epitope presentation, leading to an adverse immune response. Hence the control and avoidance of aggregation in the manufacturing process are needed because aggregates affect drug performance and safety. Hence these protein aggregates in biologic drug substances and drug products are typically considered as critical quality attributes. In addition, the identification and quantification of such protein aggregates, particularly in the presence of large concentrations of the product protein, can be challenging. Therefore use of multiple analytical methods and purification techniques is often required. Present inventors have surprisingly found that purification of final drug conjugated antibody by ion exchange chromatography followed by optionally membrane ultrafiltration-diafiltration results in improved purity of the cytotoxic drug conjugated antibody while removing excess amount of HMW species variants. Therefore, the process according to the present invention is efficient for commercial production of the drug conjugated antibody.

### SUMMARY OF THE INVENTION

The invention provides process for preparing cytotoxic drug conjugating to the linker modified antibody comprising performing the cytotoxic drug conjugation reaction at lower temperature than ambient and/or at lower pH than neutrality. The process according to the present invention comprises conjugating the linker modified antibody with a cytotoxic drug at a temperature lower than 20° C. and at a pH lower than 6.0 to covalently attach a cytotoxic drug to the linker modified antibody and thereby prepare a cytotoxic drug conjugated antibody.

In one aspect, the current invention provides a process for preparing a conjugate comprising a linker modified antibody chemically coupled to a cytotoxic drug, which comprises (a) Preparation of antibody, (b) Modification of antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products, (c) Subjecting the reaction mixture I to membrane ultrafiltration-diafiltration, selective precipitation, ion-exchange chromatography, membrane chromatography, or a combination thereof and thereby prepare a purified linker modified antibody from reaction mixture I, (d) Conjugating a cytotoxic drug to the purified linker modified antibody obtained from step (c) by reacting the linker modified antibody with a cytotoxic drug in a solution having a pH lower than 6.0 and/or at a temperature lower than 20° C. to prepare a reaction mixture II comprising (i) cytotoxic drug chemically conjugated to linker modified antibody (ii) free cytotoxic drug, (iii) unmodified antibody (iv) unconjugated linker modified antibody and/or (v) reactants and/or other by-products, (e) Subjecting the reaction mixture II to ion-exchange chromatography to purify the cytotoxic drug conjugated antibody from the other components of the reaction mixture II and thereby produce a purified cytotoxic drug conjugated antibody preparation and optionally, (f) Subjecting the purified cytotoxic drug conjugated antibody preparation obtained from step (e) to membrane ultrafiltration diafiltration.

In one of the aspects, preparation of antibody step comprises (i) purification of antibody and (ii) reconditioning of antibody. Reconditioning of antibody comprises steps of column chromatography and/or membrane ultrafiltration-diafiltration.

In other aspect, the current invention provides a process for preparing a conjugate comprising a modified antibody chemically coupled to a cytotoxic drug, which comprises following steps;
(a) Preparation of antibody comprising i. Purification of antibody and ii. Reconditioning of antibody.
(b) Modification of antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I obtained from step (b) to membrane ultrafiltration-diafiltration and thereby prepare a purified linker modified antibody from reaction mixture I.
(d) Conjugating a cytotoxic drug to the linker modified antibody in the reaction mixture I obtained from step (c) by reacting the linker modified antibody with a cytotoxic drug in a solution having a pH lower than 6.0 and at a temperature lower than 20° C. to prepare a reaction mixture II comprising (i) cytotoxic drug chemically conjugated to linker modified antibody, (ii) free cytotoxic drug, (iii) unmodified antibody, (iv) unconjugated linker modified antibody, and/or (v) reactants and/or other by-products.
(e) Subjecting the reaction mixture II obtained from step (d) to ion exchange chromatography to purify the cytotoxic drug conjugated antibody from the other components of the reaction mixture II and thereby produce a purified cytotoxic drug conjugated antibody preparation, and optionally,
(f) Subjecting the reaction mixture II obtained from step (e) to membrane ultrafiltration-diafiltration.

In another aspect, according to present invention, purification of antibody can be done by processes known in the art such as selective precipitation, chromatography techniques, filtration techniques etc. Reconditioning of antibody comprises steps of column chromatography and/or membrane ultrafiltration-diafiltration.

In one of the aspects, column chromatography step of reconditioning process step is ion-exchange chromatography.

In another aspect, the reaction mixture II is subjected to ion-exchange chromatography according to the present invention followed by membrane ultrafiltration-diafiltration.

In yet another aspect, ion exchange chromatography according to the present invention is cation exchange chromatography or anion exchange chromatography, preferably cation exchange chromatography.

In another aspect, cation exchange chromatography is performed in flow-through-and-wash mode as well as bind-elute mode.

In a preferred aspect, cation exchange chromatography after reaction mixture II preparation step is performed in flow-through-and-wash mode.

In a preferred aspect, the current invention provides a process for preparing a conjugate comprising a linker modified antibody chemically coupled to a cytotoxic drug, which comprises following steps sequentially;
(a) Preparation of antibody comprising i. Purification of antibody and ii. Reconditioning of antibody wherein reconditioning of antibody further comprises steps of cation-exchange chromatography and membrane ultrafiltration-diafiltration.
(b) Modification of antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I obtained from step (b) to membrane ultrafiltration-diafiltration and thereby prepare a purified linker modified antibody from reaction mixture I,
(d) Conjugating a cytotoxic drug to the linker modified antibody in the reaction mixture I obtained from step (c) by reacting the linker modified antibody with a cytotoxic drug in a solution having a pH lower than 6.0 and at a temperature lower than 20° C. to prepare a reaction mixture II comprising (i) cytotoxic drug chemically conjugated to linker modified antibody, (ii) free cytotoxic drug, (iii) unmodified antibody, (iv) unconjugated linker modified antibody, and/or (v) reactants and/or other by-products.
(e) Subjecting the reaction mixture II obtained from step (d) to cation exchange chromatography, wherein the drug conjugated antibody is recovered in the flow-through-and-wash of the said chromatography and optionally,
(f) Subjecting the reaction mixture II obtained from step (e) to membrane ultrafiltration-diafiltration.

According to the present invention, antibody-drug conjugate and drug conjugated antibody can be used interchangeably.

The present invention also includes a conjugate comprising an antibody chemically coupled to a cytotoxic drug prepared according to the processes described herein.

### BRIEF DESCRIPTION OF THE FIGURES

FIG. 1: It depicts the percentage purity and the level of HMW species in T-DM1 preparation generated with Example 1 as assessed by analytical HP-SEC. LMW species was not detected in the preparation.

FIG. 2: It depicts the percentage purity and the level of HMW and LMW species in T-DM1 preparation generated with Example 2 as assessed by analytical HP-SEC. Very low level of LMW species was detected in the preparation.

FIG. 3: It depicts the percentage purity and the level of HMW species in T-DM1 preparation generated with Example 3 as assessed by analytical HP-SEC. LMW species was not detected in the preparation.

### DETAILED DESCRIPTION OF THE INVENTION

In one embodiment, the process according to the present invention comprises conjugating linker modified antibody with a cytotoxic drug at a temperature lower than 20° C. and at a pH lower than 6.0 to covalently attach the cytotoxic drug to linker modified antibody and thereby prepare a drug conjugated antibody.

In a preferred embodiment, the current invention provides a process for preparing a cytotoxic drug conjugated antibody, which comprises contacting a modified antibody with a cytotoxic drug at a temperature lower than 20° C. to covalently attach a drug to the linker modified antibody and thereby prepare a drug conjugated antibody. For example, the inventive process comprises conjugating a linker modified antibody with a drug at a temperature of about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C.

In one embodiment, the process according to the present invention comprises conjugating a linker modified antibody with a cytotoxic drug at a temperature of about −10° C. to about 20° C., about 0° C., to about 20° C., about 0° C., to about 15° C., about 0° C. to about 10° C., about 0° C. to about 5° C., about 5° C. to about 15° C., about 10° C. to about 15° C., or about 5° C. to about 10° C.

In another embodiment, the inventive process comprises contacting a linker modified antibody with a cytotoxic drug at a temperature of about 15° C. (e.g., a temperature of 13° C. to 17° C. or a temperature of 14° C. to 16° C.).

In one embodiment, the process according to the present invention comprises conjugating a linker modified antibody with a cytotoxic drug in a solution having a pH of about 6.0 or less. For example, the inventive process comprises contacting a linker modified antibody with a drug in a solution having a pH about 6.0, about 5.9, about 5.8, about 5.7, about 5.6, about 5.5, about 5.4, about 5.3, about 5.2, about 5.1, about 5.0, about 4.9, about 4.8, about 4.7, about 4.6 or about 4.5.

In one embodiment, the process according to the present invention comprises conjugating a linker modified antibody with a cytotoxic drug in a solution having a pH of about 4.5 to about 6.0, about 4.7 to about 5.8, about 4.8 to about 5.4 or about 4.9 to about 5.1.

In another embodiment, the process according to the present invention comprises conjugating a linker modified antibody with a cytotoxic drug in a solution having a pH of about 5.0 (e.g., a pH of 4.8 to 5.2 or a pH of 4.9 to 5.1)

In one embodiment, the process according to the current invention comprises conjugating a linker modified antibody with a cytotoxic drug in a solution having a low pH (e.g., about 5.0 or below) at a low temperature (e.g., about 20° C. or below).

In a preferred embodiment, the process according to the current invention comprises conjugating a linker modified antibody with a cytotoxic drug in a solution having a pH about 5.0 at a temperature of about 15° C.

In accordance with the process, conjugating an antibody by a hetero-bifunctional linker produces a Reaction Mixture I comprising linker modified antibody, free linker, unmodified antibody, reactants and/or other by-products.

In accordance with the process, conjugating the linker modified antibody with a cytotoxic drug produces reaction mixture II comprising cytotoxic drug chemically conjugated to linker modified antibody, free cytotoxic drug, unmodified antibody, unconjugated linker modified antibody, reactants and/or other by-products.

A linker is “stably” bound to the antibody when the covalent bond between the linker and the antibody is not substantially weakened or severed under normal storage conditions over a period of time, which could range from a few months to a few years. In contrast, a linker is “unstably” bound to the antibody when the covalent bond between the linker and the antibody is substantially weakened or severed under normal storage conditions over a period of time, which could range from a few months to a few years.

Any suitable adsorptive chromatography matrix can be utilized for purification of linker modified antibody. Preferred adsorptive chromatography matrices include hydroxyapatite chromatography, hydrophobic charge induction chromatography (HCIC), hydrophobic interaction chromatography (HIC), ion exchange chromatography, mixed mode ion exchange chromatography, immobilized metal affinity chromatography (IMAC), dye ligand chromatography, affinity chromatography, reversed phase chromatography, and combinations thereof.

In one embodiment, the current invention provides a process for preparing a conjugate comprising an antibody chemically coupled to a cytotoxic drug, which process comprises
(a) Preparation of antibody,
(b) Modification of antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I to membrane ultrafiltration-diafiltration, selective precipitation, ion-exchange chromatography, membrane chromatography, or a combination thereof and thereby prepare a purified linker modified antibody from reaction mixture I,
(d) Conjugating a cytotoxic drug to the purified linker modified antibody obtained from step (c) by reacting the linker modified antibody with a cytotoxic drug in a solution having a pH lower than 6.0 and/or at a temperature lower than 20° C. to prepare a reaction mixture II comprising (i) cytotoxic drug chemically conjugated to linker modified antibody (ii) free cytotoxic drug, (iii) unmodified antibody (iv) unconjugated linker modified antibody and/or (v) reactants and/or other by-products,
(e) Subjecting the reaction mixture II to ion-exchange chromatography to purify the cytotoxic drug conjugated antibody from the other components of the reaction mixture II and thereby produce a purified cytotoxic drug conjugated antibody preparation, and optionally.
(f) Subjecting purified cytotoxic drug conjugated antibody preparation obtained from step (e) to membrane ultrafiltration diafiltration.

In other embodiment, preparation of antibody comprising (a) Purification of antibody and (b) Reconditioning of antibody comprising steps of column chromatography and/or membrane ultrafiltration-diafiltration.

In other embodiment, the current invention provides a process for preparing a conjugate comprising a linker modified antibody chemically coupled to a cytotoxic drug, which comprises following steps sequentially;
(a) Preparation of antibody comprising i. Purification of antibody and ii. Reconditioning of antibody,
(b) Modification of antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I obtained from step (b) to membrane ultrafiltration-diafiltration and thereby prepare a purified reaction mixture I of linker modified antibody,
(d) Conjugating a cytotoxic drug to the purified linker modified antibody obtained from step (c) by reacting the linker modified antibody with a cytotoxic drug in a solution having a pH lower than 6.0 and/or at a temperature lower than 20° C. to prepare a reaction mixture II comprising (i) cytotoxic drug chemically conjugated to linker modified antibody (ii) free cytotoxic drug, (iii) unmodified antibody (iv) unconjugated linker modified antibody and/or (v) reactants and/or other by-products,
(e) Subjecting the reaction mixture II to ion-exchange chromatography to purify the cytotoxic drug conjugated antibody from the other components of the reaction mixture II and thereby produce a purified cytotoxic drug conjugated antibody preparation and optionally,
(f) Subjecting purified cytotoxic drug conjugated antibody preparation obtained from step (e) to membrane ultrafiltration diafiltration.

In one of the embodiments, purification of antibody can be done by processes known in the art such as selective precipitation, chromatography techniques, filtration techniques etc. Reconditioning of antibody comprises steps of column chromatography and/or membrane ultrafiltration-diafiltration.

In one of the embodiments, column chromatography step of reconditioning process step is ion-exchange chromatography.

In another embodiment, the reaction mixture II is subjected to ion-exchange chromatography according to the present invention, followed by membrane ultrafiltration-diafiltration.

In yet another embodiment, ion exchange chromatography is cation exchange chromatography or anion exchange chromatography, preferably cation exchange chromatography.

In another embodiment, cation exchange chromatography is performed in flow-through-and-wash mode as well as in bind-elute mode.

In a preferred embodiment, cation exchange chromatography after reaction mixture II preparation step is performed in flow-through-and-wash mode.

In a preferred embodiment, the current invention provides a process for preparing a conjugate comprising a modified antibody chemically coupled to a cytotoxic drug, which comprises following steps sequentially;
(a) Preparation of antibody comprising i. Purification of antibody and ii. Reconditioning of antibody wherein reconditioning of antibody further comprises steps of cation-exchange chromatography and membrane ultrafiltration-diafiltration.
(b) Modification of antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I obtained from step (b) to membrane ultrafiltration-diafiltration and thereby prepare a purified reaction mixture I of linker modified antibody,
(d) Conjugating a cytotoxic drug to the purified linker modified antibody obtained from step (c) by reacting the linker modified antibody with a cytotoxic drug in a solution having a pH lower than 6.0 and/or at a temperature lower than 20° C. to prepare a reaction mixture II comprising (i) cytotoxic drug chemically conjugated to linker modified antibody (ii) free cytotoxic drug, (iii) unmodified antibody (iv) unconjugated linker modified antibody and/or (v) reactants and/or other by-products.
(e) Subjecting the reaction mixture II obtained from step (d) to cation exchange chromatography, wherein the drug conjugated antibody is recovered in the flow-through-and-wash of the said chromatography and optionally.
(f) Subjecting the reaction mixture II obtained from step (e) to membrane ultrafiltration-diafiltration.

Any suitable buffering agent can be used according to the present invention. Suitable buffering agents include, but are not limited to for example, a citrate buffer, an acetate buffer, a succinate buffer, a tris buffer and a phosphate buffer. In a preferred embodiment, the buffering agent is selected from the group consisting of phosphate buffer, succinate buffer, citrate buffer, citrate-phosphate buffer, tris buffer and a combination thereof.

According to present invention, membrane ultrafiltration-diafiltration is performed in ultrafiltration diafiltration medium selected from a phosphate buffer, a succinate buffer, a citrate buffer, an acetate buffer, a citrate-phosphate buffer, tris buffer, water and a combination thereof.

In one of the embodiment, the antibody is selected from anti-HER antibody, anti-CD20 antibody etc.

In a preferred embodiment, the antibody is selected from trastuzumab, pertuzumab rituximab etc.

In a more preferred embodiment, the antibody according to the present invention is trastuzumab.

In one of the embodiments, the drug according to the present invention is a cytotoxic drug.

In a preferred embodiment, the cytotoxic drug according to the present invention is maytansinoid, preferably DM1.

In a preferred embodiment, the drug conjugated antibody is T-DM1.

In one embodiment, cation exchange matrices can be selected from Sartobind® S, Mustang® S, SP-5PW, SP sepharose, MonoS®, Bio-rex™ 70, CM sepharose, Fractogel® EMD SO₃, CM Ceramic HyperD® F and the like.

In one embodiment, linkers can be selected from cleavable linker and non-cleavable linker, preferably non-cleavable linker.

In a preferred embodiment, non-cleavable linker is Succinimidyl-4-(N-maleimidomethyl) cyclohexane-1-carboxylate (SMCC).

### Definitions

In order that the present invention may be more readily understood, certain terms are first defined.

Antibody-drug conjugates which are useful for the treatment of cancer and other diseases are commonly composed of three distinct elements: an antibody; a linker; and a drug. Examples of such Antibody-drug conjugates of antibodies such as ErbB receptor targeting antibodies, preferably anti-HER2 or anti-HER3 targeting antibody with cytotoxic drug such as maytansinoid, taxol, auristatin, conjugates of antibodies or peptides such as hormones, trasnferrins, lipocalins with cytotoxic drug such as maytansinoid, taxol, auristatin, etc. Components of antibody-drug conjugates are defined herein below but this definition is non-limiting to the present invention.

### Antibody

The effectiveness of the compounds of the invention as therapeutic agents depends on the careful selection of an appropriate antibody. Antibody may be of any kind presently known, or that become known and includes peptides and non-peptides. Generally, these can be antibodies (especially monoclonal antibodies), lymphokines, hormones, growth factors, vitamins, nutrient-transport molecules (such as transferrin), or any other cell-binding molecule or substance that specifically binds a target. More specific examples of antibodies that can be used include: polyclonal and monoclonal antibodies, including fully human antibodies; single chain antibodies (polyclonal and monoclonal); fragments of antibodies (polyclonal and monoclonal) such as Fab, Fab′, F(ab′)2, and Fv., chimeric antibodies and antigen-binding fragments thereof; domain antibodies (dAbs) and antigen-binding fragments thereof, including camelid antibodies, shark antibodies called new antigen receptors (IgNAR) interferons (e.g. alpha, beta, gamma); lymphokines such as IL-2, IL-3, IL-4, IL-6; hormones such as insulin, TRH (thyrotropin releasing hormone), MSH (melanocyte-stimulating hormone), steroid hormones, such as andrógens and estrogens; growth factors and colony-stimulating factors such as EGF, TGF-alpha, FGF, VEGF, G-CSF, M-CSF and GM-CSF, transferrin, human tear lipocalin or its muteins and vitamins, such as folate.

### Linker

The term “hetero-bifunctional linker” refers to any chemical moiety that links a drug covalently to an antibody. In some instances, part of the linker is provided by the drug. Therefore the final linker is assembled from two pieces, the cross-linking reagent introduced into the antibody and the side chain from the drug. Linkers may broadly be either a cleavable linker or a non-cleavable linker.

Cleavable linkers are linkers that can be cleaved under mild conditions, i.e. conditions under which the activity of the maytansinoid drug is not affected. Many known linkers fall in this category and are described below:

i) Disulfide containing linkers are linkers cleavable through disulfide exchange, which can occur under physiological conditions.
ii) Acid-labile linkers are linkers cleavable at acid pH. For example, certain intracellular compartments, such as endosomes and lysosomes, have an acidic pH (pH 4-5), and provide conditions suitable to cleave acid-labile linkers.
iii) Linkers that are photo-labile are useful at the body surface and in many body cavities that are accessible to light. Furthermore, infrared light can penetrate tissue. Some linkers can be cleaved by peptidases. Only certain peptides are readily cleaved inside or outside cells, see e.g. Trouet et al., 79 Proc. Natl. Acad. Sci. USA. 626-629 (1982) and Umemoto et al. 43 Int. J. Cancer, 677-684 (1989). Furthermore, peptides are composed of α-amino acids and peptidic bonds, which chemically are amide bonds between the carboxylate of one amino acid and the α-amino group of a second amino acid. Other amide bonds, such as the bond between a carboxylate and the ε-amino group of lysine, are understood not to be peptidic bonds and are considered non-cleavable. iv) Some linkers can be cleaved by esterases. Again only certain esters can be cleaved by esterases present inside or outside cells. Esters are formed by the condensation of a carboxylic acid and an alcohol. Simple esters are esters produced with simple alcohols, such as aliphatic alcohols, and small cyclic and small aromatic alcohols.

A non-cleavable linker is any chemical moiety that is capable of linking a maytansinoid to an antibody in a stable, covalent manner and does not fall under the categories listed above as cleavable linkers. Thus, non-cleavable linkers are substantially resistant to acid-induced cleavage, light-induced cleavage, peptidase-induced cleavage, esterase-induced cleavage, and disulfide bond cleavage.

Substantially resistant” to cleavage means that the chemical bond in the linker or adjoining the linker in at least 80%, preferably at least 85%, more preferably at least 90%, even more preferably at least 95%, and most preferably at least 99% of the antibody maytansinoid conjugate population remains non-cleavable by an acid, a photolabile-cleaving agent, a peptidase, an esterase, or a chemical or a physiological compound that cleaves the chemical bond (such as a disulfide bond) in a cleavable linker, for within a few hours to several days of treatment with any of the agents described above.

Furthermore, “non-cleavable” refers to the ability of the chemical bond in the linker or adjoining to the linker to withstand cleavage induced by an acid, a photolabile-cleaving agent, a peptidase, an esterase, or a chemical or a physiological compound that cleaves a disulfide bond, at conditions under which the maytansinoid or the cell binding agent does not lose its activity.

A person of ordinary skill in the art would readily distinguish non-cleavable from cleavable linkers.

### Cytotoxic Drug

Suitable cytotoxic drugs may include but are not limited to a maytansinoid Cisplatin, Carboplatin, Oxaliplatin, 5-Fluorouracil, Taxotere (Docetaxel), Paclitaxel, Anthracycline (Doxorubicin), Methotrexate, Vinblastin, Vincristine, Vindesine, Vinorelbine, Dacarbazine, Cyclophosphamide, Etoposide, Adriamycine, Camptotecine, Combretatastin A-4 related compounds, sulfonamides, oxadiazolines, benzo[b]thiophenessynthetic spiroketal pyrans, monotetrahydrofuran compounds, curacin and curacin derivatives, methoxyestradiol derivatives, CC-1065, CC-1065 analogs and Leucovorin.

As used herein, the terms “ultrafiltration” or “UF” refers to any technique in which a solution or a suspension is subjected to a semi-permeable membrane that retains macromolecules while allowing solvent and small solute molecules to pass through. Ultrafiltration may be used to increase the concentration of macromolecules in a solution or suspension. In a preferred embodiment, ultrafiltration is used to increase the concentration of an antibody in water.

As used herein, the term “linker modified antibody” refers to a hetero-bifunctional linker which binds to antibody with one of the reactive groups, whereas the other reactive group of the linker molecule remains unaltered or intact.

As used herein, the term “diafiltration” or “DF” is used to mean a specialized class of filtration in which the retentate is diluted with solvent and re-filtered, to reduce the concentration of soluble permeates components. Diafiltration may or may not lead to an increase in the concentration of retained components, including, for example, antibodies. For example, in continuous diafiltration, a solvent is continuously added to the retentate at the same rate as the filtrate is generated. In this case, the retentate volume and the concentration of retained components do not change during the process. On the other hand, in discontinuous or sequential dilution diafiltration, an ultrafiltration step is followed by the addition of solvent to the retentate side; if the volume of solvent added to the retentate side is not equal or greater to the volume of filtrate generated, then the retained components will have a high concentration. Diafiltration may be used to alter the pH, ionic strength, salt composition, buffer composition, or other properties of a solution or suspension of macromolecules.

As used herein, the terms “ultrafiltration-diafiltration” or “UF/DF” refer to any process, technique or combination of techniques that accomplishes ultrafiltration and/or diafiltration, either sequentially or simultaneously.

The terms “ultrafiltration-diafiltration” and “membrane ultrafiltration-diafiltration” can be used interchangeably according to the present invention.

As used herein, the term “diafiltration step” refers to a total volume exchange during the process of diafiltration.

As used herein “modification step” refers to modification of antibody with linker which results into linker modified antibody.

As used herein “conjugation step” or “conjugating” refers to contacting of antibody or modified antibody to a drug. Conjugation step according to the present invention is a second step where mixture comprises linker modified antibody in a first step (modification step) and a drug. In one-step process conjugation step is the only step where mixture comprises an antibody and a drug together which is subjected to linker.

Reaction Mixture I:

Reaction Mixture I comprising the modified antibody where antibody is modified with suitable linker, free linker, unmodified antibody as well as reactants and/or other by-products. According to the present invention preferred but not limited to, reaction mixture I comprising (i) SMCC linker modified trastuzumab, (ii) free SMCC linker. (iii) unmodified trastuzumab and/or (iv) reactants and/or other by-products.

Reaction Mixture II:

Reaction mixture II comprising cytotoxic drug chemically conjugated to linker modified antibody, free cytotoxic drug, unmodified antibody, unconjugated modified antibody, and/or reactants and/or other by-products. According to the present invention preferred but not limited to, reaction mixture II comprising (i) DM1 chemically conjugated to trastuzumab through the SMCC linker, (ii) free DM1, (iii) Unmodified trastuzumab (iv) unconjugated SMCC linker modified trastuzumab and/or (iv) reactants and/or reaction by-products.

The term “T-DM1” is trastuzumab maytansinoid conjugate which is known as trastuzumab emtansine. Trastuzumab emtansine (it is referred to as T-DM1 or trastuzumab-MCC-DM1) is the DM1 covalently bound to trastuzumab antibody. In T-DM1, this antibody is linked to a hetero-bifunctional linker, succinimidyl trans-4-[maleimidylmethyl]cyclohexane-1-carboxylate (SMCC). The other end of the SMCC linker molecule is covalently bound to DM1 by a labile thioether bond. The linker binds to antibody predominantly at lysine residues with a net stoichiometry of DM1 to antibody of approximately 3.5. The resulting compound is referred to as a drug conjugated antibody or ADC.

Ion exchange chromatography is a chromatographic technique that is commonly used for the purification of antibodies. In ion exchange chromatography, charged patches on the surface of the solute are attracted by opposite charges attached to a chromatography matrix, provided the ionic strength of the surrounding buffer is low. Elution is generally achieved by increasing the ionic strength (i.e. conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). In the past, these changes have been progressive; i.e., the pH or conductivity is increased or decreased in a single direction.

A “cation exchange matrix” refers to a solid phase which is negatively charged, and which thus can exchange with free cations in an aqueous solution passed over or through the solid phase. A negatively charged ligand attached to the solid phase to form the cation exchange matrices may, e.g., be a carboxylate or sulfonate. Commercially available cation exchange matrices include Sartobind S, Mustang S, SP-5PW, MonoS, Bio-rex70, CM sepharose, Fractogel® EMD SO₃, CM Ceramic HyperD® F, carboxy-methyl-cellulose, BAKERBOND ABX™, sulphopropyl (SP) immobilized on agarose (e.g. SP-SEPHAROSE FAST FLOW™ or SP-SEPHAROSE HIGH PERFORMANCE™, from Pharmacia) and sulphonyl immobilized on agarose (e.g. S-SEPHAROSE FAST FLOW™ from Pharmacia) and the like.

The term “anion exchange matrix” is used to refer to a solid phase which is positively charged, e.g. having one or more positively charged ligands, such as quaternary amino groups, attached thereto. Commercially available anion exchange matrices include DEAE cellulose. QAE SEPHADEX™ and FAST Q SEPHAROSE™ (Pharmacia).

Size Exclusion Chromatography:

In size exclusion chromatography, the separation of components is a function of their molecular size and the stationary phase typically does not attract the components. Separation depends on the amount of time that the substances spend in the porous stationary phase as compared to time in the fluid. The probability that a molecule will reside in a pore depends on the size of the molecule and the pore. In the biological sciences, size exclusion chromatography is used for the isolation and purification of biological molecules, such as peptides, hormones or DNA.

Selective precipitation is a technique of separating ions in an aqueous solution by using a reagent that precipitates one or more of the ions, while leaving other ions in solution.

“Cross-linked species” as used herein is the non-reducible species of the conjugates comprising the light-light chains, light-heavy chains, heavy-heavy chains etc.

The abbreviations used in the present description are defined below:
HP-SEC: High performance-size exclusion chromatography.
MWCO: Molecular weight cut-off.

### CE-SDS: Capillary Electrophoresis-Sodium Dodecyl Sulfate

UF/DF: ultrafiltration/diafiltration
HMW species: High Molecular Weight species
LMW species: Low Molecular Weight species

### MCB: Master Cell Bank

### WCB: Working Cell Bank

### CHO: Chinese Hamster Ovary

### EXAMPLES

The following examples further illustrate the invention, but do not limit its scope in any way.

### Example 1: Synthesis of Trastuzumab-Maytansinold Conjugate (T-DM1) at pH 5.0 and Temperature Below 23° C. Conditions

Following steps are involved in the synthesis of trastuzumab-maytansinoid conjugate:

### Fermentation and Purification Process

Trastuzumab is produced by suspension CHO cell culture process in fed-batch mode in bioreactor. Every batch production in upstream starts with the revival of a MCB or WCB vial of CHO cells harboring the trastuzumab gene. Following revival of the vial content, CHO cell culture process undergoes a series of seed development steps to generate adequate number of cells prior to inoculation in the production bioreactor. Cell culture process in the production bioreactor is carried with a series of pre-defined process parameters to express the trastuzumab in soluble form. At the end of cell culture process, the batch is harvested and the harvested cell culture fluid is processed by using conventional centrifugation, membrane filtration and chromatography techniques to obtain the purified trastuzumab.

### Reconditioning of Trastuzumab

Formulated drug substance of trastuzumab (about 21 mg/mL) was diluted up to 6-fold with 50 mM potassium phosphate buffer of pH 5.0 and pH of the diluted antibody solution was further adjusted to pH 5.0 with the addition of ortho-phophoric acid. The mixture was passed through a 0.22 μm filter and loaded on to a cation exchange column (SP-sepharose), at 115 cm/h linear flow rate, equilibrated with 50 mM of potassium phosphate buffer pH 5.0. This column step is performed for deformulation of trastuzumab antibody. Upon loading on to the column, trastuzumab antibody was eluted with 50 mM potassium phosphate buffer of pH 6.5 containing 100 mM NaCl, in a linear gradient fashion. Trastuzumab antibody was collected in the form of a single peak eluted out of the ion exchange column.

Other cation exchange matrices, e.g. CM Sepharose, SP5PW, Fractogel® EMD SO₃, Sartobind S, Mustang S, CM Ceramic HyperD® F, can also be used for deformulation of trastuzumab antibody at this step. Trastuzumab antibody recovered from the cation exchange column was subjected to membrane ultrafiltration-diafiltration in the presence of 50 mM potassium phosphate buffer of pH 6.5 containing 50 mM NaCl and 2 mM EDTA using 30 kDa MWCO membrane to tune up to the next reaction step. The diafiltered antibody solution was passed through a 0.22 μm filtration and was subjected to covalent modification by SMCC.

### Covalent Modification of Trastuzumab by SMCC

After reconditioning, trastuzumab antibody, at about 10 mg/mL, was treated with about 7 molar excess of SMCC (trans-succinimidyl 4(N-maleimidomethyl) cyclohexane-1-carboxylate), a hetero-bifunctional linker, at about 25° C. for a maximum period of 2 hours with gentle mixing. Modification reaction was conducted at about pH 6.5. At the end of the reaction, the mixture was passed through a 0.22 μm filter. At this step the modified trastuzumab is termed as T-MCC. This reaction mixture is termed as Reaction Mixture I.

Removal of Excess Unreacted SMCC and Reaction by-Products by Membrane Ultrafltration-Diafltration

Removal of excess, unreacted SMCC and reaction by-products from the Reaction Mixture I can be performed either by column chromatography or by membrane ultrafiltration-diafiltration. In this case, membrane ultrafiltration-diafiltration was carried out at a temperature between 15° C. and 20° C. by using 30 kDa MWCO membrane in the presence of 50 mM sodium succinate buffer of pH 5.0 containing 50 mM NaCl and 2 mM EDTA.

### Covalent Conjugation of DM1 to T-MCC

T-MCC obtained from Reaction Mixture I was subjected to conjugation reaction in the presence of about 8.5 molar excess of maytansinoid drug (DM1) over the antibody concentration. Reaction was performed at about 8 mg/mL T-MCC concentration (protein basis); temperature 15° C. for 10 hours. Conjugation reaction was performed in the presence of 50 mM sodium succinate buffer of pH 5.0 containing 50 mM NaCl, 2 mM EDTA. Conjugation reaction can also be performed in 50 mM potassium phosphate buffer of pH 5.0 containing 50 mM NaCl, 2 mM EDTA. This reaction mixture is termed as Reaction Mixture II. DM1 conjugation to trastuzumab takes place through SMCC linker molecule with the formation of covalent thioether bond. The maleimide functional group of the hetero-bifunctional linker molecule forms a thioether linkage with the free thiol (—SH) group of DM1.

At the end of conjugation reaction, the antibody-drug conjugate (T-DM1) mixture was passed through a 0.22 μm filter and subjected to membrane ultrafiltration-diafiltration for the removal of excess amount of unreacted free DM1.

Removal of Free/Unreacted DM1 from T-DM1 by Membrane Ultrafltration-Diafltration

Following the conjugation reaction, the Reaction Mixture II was subjected to membrane ultrafiltration-diafiltration in the presence of 10 mM sodium succinate buffer of pH 5.0 to remove the excess unreacted, free DM1 by using 30 kDa MWCO membrane, under ambient temperature condition.

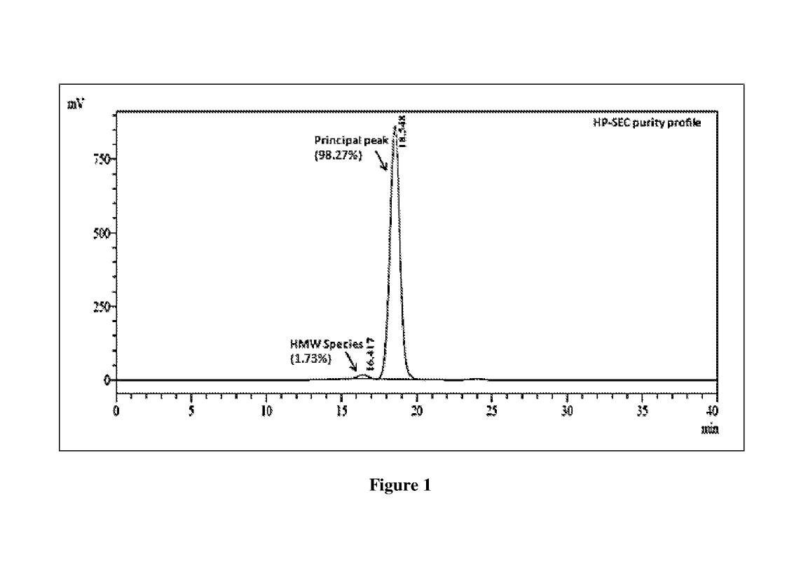

After ultrafiltration-diafiltration, T-DM1 was analyzed for purity and cross-linked species variants by HP-SEC and CE-SDS, respectively. Data are summarized in Table 1 and FIG. 1.

TABLE 1
Summary of results obtained after membrane ultrafiltration-diafiltration
of purified Trastuzumab Emtansine (T-DM1)

| Description | Parameters | Results |
|---|---|---|
| Purified | Purity by HP-SEC; native | Principal Peak: 98.27% |
| T-DM1 | condition | HMW species: 1.73% |
| | | LMW species: |
| | | Not detected |
| | Cross-linked species variants by | Total cross |
| | reducing CE-SDS; denaturing | linked species: |
| | conditions | 8.60% |

### Example 2: Synthesis of Trastuzumab-Maytansinoid Conjugate (T-DM1) at pH 6.5 and Temperature 23° C.

Following steps are involved in the synthesis of trastuzumab-maytansinoid conjugate:

### Fermentation and Purification Process

Trastuzumab is produced by suspension CHO cell culture process in fed-batch mode in bioreactor. Every batch production in upstream starts with the revival of a MCB or WCB vial of CHO cells harboring the trastuzumab gene. Following revival of the vial content, CHO cell culture process undergoes a series of seed development steps to generate adequate number of cells prior to inoculation in the production bioreactor. Cell culture process in the production bioreactor is carried with a series of pre-defined process parameters to express the trastuzumab in soluble form. At the end of cell culture process, the batch is harvested and the harvested cell culture fluid is processed by using conventional centrifugation, membrane filtration and chromatography techniques to obtain the purified trastuzumab.

### Reconditioning of Trastuzumab

Formulated drug substance of trastuzumab (about 21 mg/mL) was diluted up to 6-fold with 50 mM potassium phosphate buffer of pH 5.0 and pH of the diluted antibody solution was further adjusted to pH 5.0 with the addition of ortho-phophoric acid. The mixture was passed through a 0.22 μm filter and loaded on to a cation exchange column (SP-sepharose), at 115 cm/h linear flow rate, equilibrated with 50 mM of potassium phosphate buffer pH 5.0. This column step is performed for deformulation of trastuzumab antibody. Upon loading on to the column, trastuzumab antibody was eluted with 50 mM potassium phosphate buffer of pH 6.5 containing 100 mM NaCl, in a linear gradient fashion. Trastuzumab antibody was collected in the form of a single peak eluted out of the ion exchange column.

Other cation exchange matrices, e.g. CM Sepharose, SP5PW, Fractogel® EMD SO₃, Sartobind S, Mustang S, CM Ceramic HyperD® F, can also be used for deformulation of trastuzumab antibody at this step. Trastuzumab antibody recovered from the cation exchange column was subjected to membrane ultrafiltration-diafiltration in the presence of 50 mM potassium phosphate buffer of pH 6.5 containing 50 mM NaCl and 2 mM EDTA using 30 kDa MWCO membrane to tune up to the next reaction step. The diafiltered antibody solution was passed through a 0.22 μm filtration and was subjected to covalent modification by SMCC.

### Covalent Modification of Trastuzumab by SMCC

After reconditioning, trastuzumab antibody, at about 15 mg/mL, was treated with about 7 molar excess of SMCC (trans-succinimidyl 4(N-maleimidomethyl) cyclohexane-1-carboxylate), a hetero-bifunctional linker, at about 25° C. for a maximum period of 2 hours with gentle mixing. Modification reaction was conducted at pH 6.5. At the end of reaction, the reaction mixture was passed through a 0.22 μm filter. At this step the modified trastuzumab is termed as T-MCC. This reaction mixture is termed as Reaction Mixture I.

Removal of Excess Unreacted SMCC and Reaction by-Products by Gel Filtration Column Chromatography

Removal of excess, unreacted SMCC and reaction by-products from the Reaction Mixture I can be performed either by column chromatography or by membrane ultrafiltration-diafiltration. In this case, gel filtration column chromatography (Sephadex G25) was carried out at 350 mL/min volumetric flow rate, under ambient temperature conditions. Column was equilibrated with 50 mM potassium phosphate buffer of pH 6.5 containing 50 mM NaCl and 2 mM EDTA. The modified trastuzumab antibody was recovered from the column with a single peak.

### Covalent Conjugation of DM1 to T-MCC

T-MCC obtained from Reaction Mixture I was subjected to conjugation reaction in the presence of about 8.5 molar excess of maytansinoid drug (DM1) over the antibody concentration. Reaction was performed at about 10 mg/mL of T-MCC (protein basis) 23° C. for 16.5 hours. Conjugation reaction was performed in the presence of 50 mM potassium phosphate buffer of pH 6.5 containing 50 mM NaCl and 2 mM EDTA. This reaction mixture is termed as Reaction Mixture II. At the end of conjugation reaction, the antibody-drug conjugate (T-DM1) mixture was passed through a 0.22 μm filter and subjected to membrane ultrafiltration-diafiltration for the removal of excess amount of unreacted free DM1.

Removal of Free/Unreacted DM1 from T-DM1 by Membrane Ultrafiltration-Diafiltration

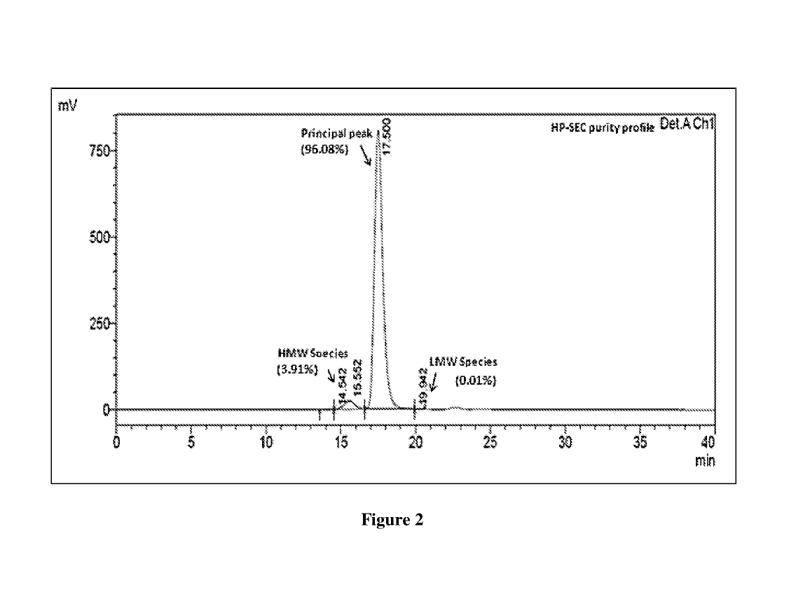

Following the conjugation reaction, the Reaction Mixture II was subjected to membrane ultrafiltration-diafiltration in the presence of 10 mM succinate buffer of pH 5.0 to remove the excess unreacted, free DM1 by using 30 kDa MWCO membrane, under ambient temperature conditions. T-DM1 was analyzed for purity and cross-linked species variants by HP-SEC and CE-SDS, respectively. Data are summarized in Table 2 and FIG. 2.

TABLE 2
Summary of results obtained after membrane ultrafiltration-diafiltration
of purified Trastuzumab Emtansine (T-DM1)

| Description | Parameters | Results |
|---|---|---|
| Purified T- | Purity by HP-SEC; native | Principal peak: 96.08% |
| DM1 | condition | HMW species: 3.91% |
| | | LMW species: 0.01% |
| | Cross-linked species variants | Total cross linked |
| | by reducing CE-SDS; | species: 13.67% |
| | denaturing conditions | |

### Example 3: Synthesis of Trastuzumab-Maytansinold Conjugate (T-DM1) at pH 5.0, Temperature 15° C. Followed by Purification of T-DM1 Conjugate Using Cation Exchange Chromatography

Following steps are involved in the synthesis of trastuzumab-maytansinoid conjugate:

### Fermentation and Purification Process

Trastuzumab is produced by suspension CHO cell culture process in fed-batch mode in bioreactor. Every batch production in upstream starts with the revival of a MCB or WCB vial of CHO cells harboring the trastuzumab gene. Following revival of the vial content, CHO cell culture process undergoes a series of seed development steps to generate adequate number of cells prior to inoculation in the production bioreactor. Cell culture process in the production bioreactor is carried with a series of pre-defined process parameters to express the trastuzumab in soluble form. At the end of cell culture process, the batch is harvested and the harvested cell culture fluid is processed by using conventional centrifugation, membrane filtration and chromatography techniques to obtain the purified trastuzumab.

### Reconditioning of Trastuzumab

Formulated drug substance of trastuzumab (about 21 mg/mL) was diluted up to 6-fold with 50 mM potassium phosphate buffer of pH 5.0 and pH of the diluted trastuzumab antibody solution was further adjusted to pH 5.0 with the addition of ortho-phophoric acid. The mixture was passed through a 0.22 μm filter and loaded on to a cation exchange column (SP-sepharose), at 115 cm/h linear flow rate, equilibrated with 50 mM of potassium phosphate buffer pH 5.0. This column step is performed for deformulation of trastuzumab antibody. Upon loading on to the column, trastuzumab antibody was eluted with 50 mM postassium phosphate buffer of pH 6.5 containing 100 mM NaCl, in a linear gradient fashion. Trastuzumab antibody was collected in the form of a single peak eluted out of the ion exchange column. Other cation exchange matrices, e.g. CM Sepharose, SP5PW, Fractogel® EMD SO₃, Sartobind, Mustang S, CM Ceramic HyperD® F, can also be used for deformulation of trastuzumab antibody at this step.

Trastuzumab antibody recovered from the cation exchange column was subjected to membrane ultrafiltration-diafiltration in the presence of 50 mM potassium phosphate buffer of pH 6.5 containing 50 mM NaCl and 2 mM EDTA using 30 kDa MWCO membrane to tune up to the next reaction step. The diafiltered antibody solution was passed through a 0.22 μm filtration and was subjected to covalent modification by SMCC.

### Covalent Modification of Trastuzumab by SMCC

After reconditioning, trastuzumab antibody, at about 10 mg/mL, was treated with about 7 molar excess of SMCC (trans-succinimidyl 4(N-maleimidomethyl) cyclohexane-1-carboxylate), a hetero-bifunctional linker, at about 25° C. for a maximum period of 2 hours with gentle mixing. Modification reaction was conducted at about pH 6.5. At the end of reaction, the reaction mixture was passed through a 0.22 μm filter. At this step the modified trastuzumab is termed as T-MCC. This reaction mixture is termed as Reaction Mixture I.

Removal of Excess Unreacted SMCC and Reaction by-Products by Membrane Ultrafiltration-Diafiltration

Removal of excess, unreacted SMCC and reaction by-products from the Reaction Mixture I can be performed either by column chromatography or by membrane ultrafiltration-diafiltration. In this case, membrane ultrafiltration-diafiltration was carried out at a temperature between 15° C.-20° C. by using 30 kDa MWCO membrane in the presence of 50 mM sodium succinate buffer of pH 5.0 containing 50 mM NaCl and 2 mM EDTA.

### Covalent Conjugation of DM1 to T-MCC

T-MCC obtained from Reaction Mixture I was subjected to conjugation reaction in the presence of about 8.5 molar excess of maytansinoid drug (DM1) over the antibody concentration. Reaction was performed at about 8 mg/mL T-MCC concentration (protein basis) and 15° C. for 9 hours. Conjugation reaction was performed in the presence of 50 mM sodium succinate buffer of pH 5.0 containing 50 mM NaCl, 2 mM EDTA. This reaction mixture is termed as Reaction Mixture II. At the end of conjugation reaction, the antibody-drug conjugate (T-DM1) mixture is termed as Reaction Mixture II was passed through a 0.22 μm filter and subjected to cation exchange chromatography.

### Purification by Cation Exchange Chromatography

After filtration, the crude Reaction Mixture II containing T-DM1 was subjected to cation exchange membrane chromatography (e.g. Sartobind S or Mustang S), mainly for the removal of excess amount of high molecular weight species variants of antibody. Membrane chromatography filter was equilibrated with 50 mM sodium succinate buffer of pH 5.0 containing 50 mM sodium chloride and 2 mM EDTA, under ambient conditions. In this chromatography process step, the desired T-DM1 molecule was recovered in the flow-through-and-wash fraction at 150 mL/min., Drug antibody conjugate (T-DMI) recovered from the ion exchange chromatography step was further subjected to membrane ultrafiltration-diafiltration for the removal of excess amount free DM1 from the mixture.

Removal of Free/Unreacted DM1 from T-DM1 by Membrane Ultrafltration-Diafltration

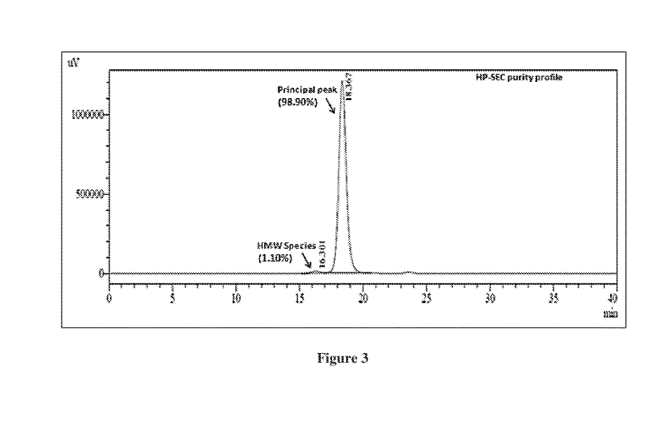

After the chromatography step, T-DM1 solution was subjected to membrane ultrafiltration-diafiltration in the presence of 10 mM sodium succinate buffer of pH 5.0 to remove the excess unreacted, free DM1 by using 30 kDa MWCO membrane, under ambient temperature conditions. T-DM1 was analyzed for, purity and cross-linked species variants by HP-SEC and CE-SDS, respectively. Data are summarized in Table 3 and FIG. 3.

TABLE 3
Summary of results obtained after cation exchange membrane
chromatography and membrane ultrafiltration-diafiltration
of purified Trastuzumab Emtansine (T-DM1)

| Description | Parameters | Results |
|---|---|---|
| Purified | Purity by HP-SEC; native | Principal peak: 98.90% |
| T-DM1 | condition | HMW species: 1.10% |
| | | LMW species: Not detected |
| | Cross-linked | Total cross linked |
| | species variants | species: 8.71% |
| | by reducing CE-SDS; | |
| | denaturing conditions | |

According to the present invention it can be concluded from the results obtained with Example 1 and 2 that conjugation of SMCC modified trastuzumab with maytansinoid derivative drug, DM1 at lower temperature than ambient and at a pH lower than 6.0 provides drug conjugated antibody, T-DM1 with desired quality containing substantially lower amount of cross-linked species. It can be further concluded from the results obtained with Example 1 and 3 that membrane chromatography provides higher level purity of T-DM1 while removing excess amount of HMW species variants.

1. A process for preparing antibody-drug conjugate comprising following steps sequentially:
(a) Preparation of antibody,
(b) Modification of the antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I to membrane ultrafiltration-diafiltration, selective precipitation, ion-exchange chromatography, membrane chromatography, or a combination thereof and thereby prepare a purified linker modified antibody from reaction mixture I,
(d) Conjugating a cytotoxic drug to the purified linker modified antibody obtained from step (c) by reacting the linker modified antibody with a cytotoxic drug in a solution having a pH lower than 6.0 and/or at a temperature lower than 20° C. to prepare a reaction mixture II comprising (i) cytotoxic drug chemically conjugated to linker modified antibody (ii) free cytotoxic drug, (iii) unmodified antibody (iv) unconjugated linker modified antibody and/or (v) reactants and/or other by-products,
(e) Subjecting the reaction mixture II to ion-exchange chromatography to purify the cytotoxic drug conjugated antibody from the other components of the reaction mixture II and thereby producing a purified cytotoxic drug conjugated antibody preparation and optionally, and
(f) Subjecting the purified cytotoxic drug conjugated antibody preparation obtained from step (e) to membrane ultrafiltration diafiltration.

2. The process as claimed in claim 1 wherein step (a) comprises i. Purification of antibody and ii. Reconditioning of the antibody.

3. The process as claimed in claim 1, wherein the preparation of the antibody-drug conjugate comprises the following steps sequentially,
(a) Preparation of antibody comprising i. Purification of the antibody and ii. Reconditioning of the antibody,
(b) Modification of the antibody by a hetero-bifunctional linker at about 25° C. to attach the linker covalently to the antibody to prepare a reaction mixture I comprising (i) linker modified antibody, (ii) free linker, (iii) unmodified antibody and/or (iv) reactants and/or other by-products,
(c) Subjecting the reaction mixture I obtained from step (b) to membrane ultrafiltration-diafiltration and thereby prepare a purified linker modified antibody from reaction mixture I,
(d) Conjugating a cytotoxic drug to the linker modified antibody in the reaction mixture I obtained from step (c) by reacting the linker modified antibody with a cytotoxic drug in a solution having a pH lower than 6.0 and at a temperature lower than 20° C. to prepare a reaction mixture II comprising (i) cytotoxic drug chemically conjugated to linker modified antibody, (ii) free cytotoxic drug, (iii) unmodified antibody, (iv) unconjugated linker modified antibody, and/or (v) reactants and/or other by-products,
(e) Subjecting the reaction mixture II obtained from step (d) to ion exchange chromatography to purify the cytotoxic drug conjugated antibody from the other components of the reaction mixture II and thereby producing a purified cytotoxic drug conjugated antibody preparation and optionally, and
(f) Subjecting the reaction mixture II obtained from step (e) to membrane ultrafiltration-diafiltration.

4. The process as claimed in claim 2, wherein reconditioning of the antibody comprises ion exchange chromatography and membrane ultrafiltration-diafiltration.

5. The process as claimed in claim 1, wherein the ion exchange chromatography is cation exchange chromatography or anion exchange chromatography.

6. The process as claimed in claim 1, wherein the ion exchange chromatography is cation exchange chromatography.

7. The process as claimed in step (e) of claim 1, wherein the ion exchange chromatography is performed in flow-through-and-wash mode.

8. The process as claimed in claim 1, wherein conjugation of linker modified antibody with cytotoxic drug is performed at a temperature of about −10° C. to about 20° C., about 0° C. to about 20° C., about 0° C. to about 15° C., about 0° C. to about 10° C., about 0° C. to about 5° C., about 5° C. to about 15° C., about 10° C. to about 15° C. or about 5° C. to about 10° C. and/or at a pH of about 4.5 to about 6.0, about 4.7 to about 5.8, about 4.8 to about 5.4 or about 4.9 to about 5.1.

9. The process as claimed in claim 8, wherein conjugation of linker modified antibody with cytotoxic drug is performed at a temperature of about 20° C., about 19° C., about 18° C., about 17° C., about 16° C., about 15° C., about 14° C., about 13° C., about 12° C., about 11° C., about 10° C., about 9° C., about 8° C., about 7° C., about 6° C., about 5° C., about 4° C., about 3° C., about 2° C., about 1° C., or about 0° C. and/or at a pH of about 6.0, about 5.9, about 5.8, about 5.7, about 5.6, about 5.5, about 5.4, about 5.3, about 5.2, about 5.1, about 5.0, about 4.9, about 4.8, about 4.7, about 4.6 or about 4.5.

10. The process as claimed in claim 1, wherein conjugation of linker modified antibody with cytotoxic drug is performed in a solution having a pH about 5.0 and/or at a temperature about 15° C.

11. The process as claimed in claim 1, wherein the solution is buffer selected from a phosphate buffer, a succinate buffer, a citrate buffer, an acetate buffer, a citrate-phosphate buffer, Tris buffer and a combination thereof.

12. The process as claimed in claim 1, wherein membrane ultrafiltration-diafiltration is performed in ultrafiltration-diafiltration medium selected from a phosphate buffer, a succinate buffer, a citrate buffer, an acetate buffer, a citrate-phosphate buffer, tris buffer, water and a combination thereof.

13. The process as claimed in claim 1, wherein the antibody is selected from anti-HER antibody and anti-CD20 antibody.

14. The process as claimed in claim 13, wherein the antibody is selected from trastuzumab, pertuzumab, and rituximab.

15. The process as claimed in claim 14, wherein antibody is trastuzumab.

16. The process as claimed in any claim 1, wherein cytotoxic drug is maytansinoid.

17. The process as claimed in claim 1, wherein the hetero-bifunctional linker is selected from cleavable linker and non-cleavable linker.

18. The process as claimed in claim 17, wherein the hetero-bifunctional linker is non-cleavable linker.

19. The process as claimed in claim 18, wherein hetero-bifunctional linker is succinimidyl-4-(N-maleimidomethyl) cyclohexane-1-carboxylate (SMCC).

20. The process as claimed in claim 1 wherein antibody-drug conjugate is T-DM1.
